(19) United States
(12) Patent Application Publication
(10) Pub. No.: US 2014/0142600 A1
(43) Pub. Date: May 22, 2014

(54) RETRACTABLE UNIVERSAL SAFETY SCALPEL

(71) Applicant: Paramount Surgimed Ltd., New Delhi (IN)

(72) Inventor: Abhay Kumar, New Delhi (IN)

(73) Assignee: Paramount Surgimed Ltd., New Delhi (IN)

(21) Appl. No.: 13/955,574

(22) Filed: Jul. 31, 2013

(30) Foreign Application Priority Data
Nov. 16, 2012 (IN) 3530/DEL/2012

(51) Int. Cl. A61B 17/3211

(52) U.S. Cl. CPC A61B 17/3211
USPC 606/167

(57) ABSTRACT

A retractable universal safety scalpel with a handle; a blade carrier element on which is secured a blade slidably mounted within the housing for exposure from the housing; slider elements secured to the blade carrier element through a recess formed in the handle in a slidable arrangement with the housing and extending externally in an axial direction of the housing in multiple positions including covered and locked positions for pre-use, in-use and post-use disposal stages.

### CROSS-REFERENCE TO RELATED APPLICATIONS

This application claims priority from Indian Patent Application No. 3530/DEL/2012 filed on Nov. 16, 2012, the entire disclosure of which is incorporated herein by reference.

### FIELD OF THE INVENTION

The present invention relates to a surgical tool and more particularly to a retractable universal safety scalpel having safety features and means movable along the scalpel handle to move the blade mounted in the said handle between a pre-use un-exposed position to in-use exposed position and to an after-use unexposed position thereby permanently covering and locking the blade.

### BACKGROUND OF THE INVENTION

Surgical cutting instruments such as scalpels are widely used to perform surgery. The disposable surgical scalpels are well known in the art and often comprise a handle, typically formed of a plastic material, to which is attached either permanently or detachably, a scalpel blade. In operation, the handle is dealt with to get the scalpel blade exposed for use. This, of course, also exposes the blade to all individuals, doctors, nurses, medical technicians, etc., associated with a surgical procedure, as well as those individuals who are involved with the disposal of the used scalpel. Even with the exercise of great care, individuals are frequently inadvertently cut by the exposed scalpel blade. The dangers of being cut and transmission of infectious diseases when cut by a used scalpel blade are thus ever-present. Thus, the individuals handling the surgical scalpel remain at risk.

Further, the potential for blood born infection has created a demand for surgical instruments which in protective position shields permanently the blade once used thereby providing protection to the user from inadvertent dangers of being cut. A wide variety of scalpels with retractable blades are available in the industry. Nevertheless, there remains a continuing need to improve the performance of such devices so that they meet the users' expectations in terms of performance as a surgical instrument as well as offer improved safety.

Moreover, there remains a constant need for a surgical scalpel which provides improved control and safety features reducing the amount of pressure required to be exerted by the surgeon to maintain control of the scalpel thereby making the handling and operation of the scalpel much easier.

### SUMMARY OF THE INVENTION

The disclosed surgical tool relates to a retractable universal safety scalpel comprising a handle having a proximal section and a distal section with a longitudinally extending bore defining a housing; a blade carrier element having mid-section, proximal end and a distal end on which is secured a blade slidably mounted within said housing being capable of getting exposed from the end of distal section of said housing; first and second slider elements secured to said blade carrier element through a recess formed in the said handle extending from the forward end closer to the said opening to the rear end closer to the distal section of the said handle in a slidable arrangement with the said housing and extending externally in an axial direction of said housing to move said blade carrier moving the said blade from an un-exposed pre-use position to an exposed in-use position and to an after-use position capable of covering and locking permanently the said blade member completely within the said housing of the said handle; a plurality of locking means is provided in the said housing to enable locking of the blade in an un-exposed pre-use position to an exposed in-use position and to an after-use position capable of covering and locking permanently the said blade member wherein the said housing being provided with lock cooperating mechanisms at three positions thereon corresponding to pre-use, in-use and post-use disposal stages, said lock cooperating mechanisms cooperating with the respective locking means provided thereon.

Further features of the invention provides for a first and second slider element movably mounted in the said handle for sliding movement of the said blade carrier element comprising the blade relative to said handle between a pre-use un-exposed position to in-use exposed position and to an after-use unexposed position allowing the said blade to move to each of said positions when the afore-said slider elements are activated.

Further features of the invention provide for the said first and second slider elements to be in a movably locking/engaging arrangement with the recess provided in the axial direction opening through and between opposite sides of the handle, said sliding members being provided with one or more engagement means, for example tooth and notch formations that cooperate to hold the said blade carrier element and slider members in one or more locking and/or engagement arrangements capable of being movable in each of the said pre-use, in-use and after-use positions such that a person operating the scalpel will know exactly, by feel and be aware of the position and movement of the blade member in the said housing of the said handle. It will also be appreciated that the engagement means being in engaging arrangement with said recess is shaped for cooperation with the margins of the said recess to maintain the slider members in positions being smoothly movable in each of the afore-said positions.

Further features of the invention provides for a retractable universal safety scalpel wherein said blade carrier element member includes a blade. Guide surfaces are provided in the inner surface of the housing of the said handle in an engaging arrangement with said blade carrier and movable along said handle in response to sliding movement of said slider elements in each of the said pre-use, in-use and after-use positions.

Further features of the invention provide for the said blade carrier of the said retractable universal safety scalpel being configured such that it can accept blades having plurality of different profiles and/or sizes thereby making it universal in use.

Further features of the invention provides for a retractable universal safety scalpel, which in use position allows for direct finger control of the scalpel handle and blade without interfering or getting interfered from the said slider elements. Moreover, the slider elements are slidable along the handle between all positions being adapted to move the slider elements between its pre-use position and in-use position. Further, the slider elements are slidable between pre-use position and in-use positions multiple times, whereby the scalpel may be used, set aside and then reused with the movable slider members.

Further features of the invention provides for a retractable universal safety scalpel wherein said slider elements are movable during the course of in-use position allowing the user to have the exposure of desired length of the surgical blade.

Further features of the invention provides for a retractable universal safety scalpel wherein said slider elements are movable into a permanent lock position covering/locking permanently the said blade in the housing of the said handle.

Further features of the invention provides for locking means in the housing to enable locking of the blade in an un-exposed pre-use position to an exposed in-use position and to an after-use position capable of covering and locking permanently the said blade member wherein the said housing being provided with lock cooperating mechanisms at three positions thereon corresponding to pre-use, in-use and post-use disposal stages, said lock cooperating mechanisms cooperating with the respective locking means provided thereon.

Further features of the invention provides for a retractable universal safety scalpel which can be easily used/handled by both a right-handed as well as left-handed person.

Further features of the invention provides for a retractable universal safety scalpel including one or more safety elements having safety features such as locking safety features to prevent completely the accidental retraction of the blade when in use and/or providing permanent lock for the blade after use.

In still other embodiments of the invention, methods of use and construction are also provided.

### BRIEF DESCRIPTION OF THE DRAWINGS

The present invention will now be explained in more detail in the following with reference to the preferred embodiments and to the accompanying drawings, wherein:

FIGS. 1 and 2 illustrate right and left side exploded perspective views of the retractable universal safety scalpel, where the assembly elements are detached from the scalpel cover according to the present invention;

FIGS. 3 and 4 illustrate right and left side exploded perspective views of the retractable universal safety scalpel, where the blade carrier element is slidably attached in the housing of the scalpel cover exposing the blade in-use position according to the present invention;

FIGS. 5 and 6 illustrate right and left side exploded perspective views of the retractable universal safety scalpel, where the first and second slider elements are movably mounted respectively in the recess of the scalpel cover exposing the blade in-use position according to the present invention;

FIGS. 7 and 8 illustrate right and left side views of the assembled retractable universal safety scalpel exposing the blade in-use position according to the present invention.

FIG. 9 illustrates the top view of the assembled retractable universal safety scalpel exposing the blade in-use position according to the present invention.

FIGS. 10 and 11 illustrate right and left side see-through views of the retractable universal safety scalpel where the blade carrier element is slidably engaged with the distal end of the scalpel handle exposing the blade in-use position according to the present invention.

FIGS. 12 and 13 illustrate right and left side views of the blade carrier element according to the present invention.

FIGS. 14 and 15 illustrate perspective views from the right and left side of the blade carrier element according to the present invention.

FIGS. 16A-16D collectively referred to as FIG. 16 illustrate right, left, front and perspective views of the first slider element in the open position according to the present invention.

FIGS. 17A-17D collectively referred to as FIG. 17 illustrate right, left, front and perspective views of the second slider element in the open position according to the present invention.

FIGS. 18A-18B collectively referred to as FIG. 18 illustrate perspective views from the right and left side and top view of the handle according to the present invention

FIGS. 19 to 21 illustrate another embodiment of the retractable universal safety scalpel including locking members to lock the blade carrier element with the blade in a locking position.

FIGS. 22 to 24 illustrate another embodiment of the slider elements of the retractable universal safety scalpel.

FIGS. 25 and 26 illustrate another embodiment of the retractable universal safety scalpel exposing the blade in-use position.

### DETAILED DESCRIPTION OF THE INVENTION

Generally speaking, the term “proximal” refers to a region of the device or parts thereof or a location on the device which is closest to, for example, a user using the device. In contrast to this, the term “distal” refers to a region of the device which is farthest from the user, for example, the distal region of a surgical scalpel will be the region forming the opening from which the blade mounted in the housing of the handle gets exposed when the scalpel is in-use position.

The term “permanently lock position” as used herein means covering the blade member of the surgical scalpel in such a manner that it cannot be un-covered or exposed without destroying the handle of the surgical scalpel.

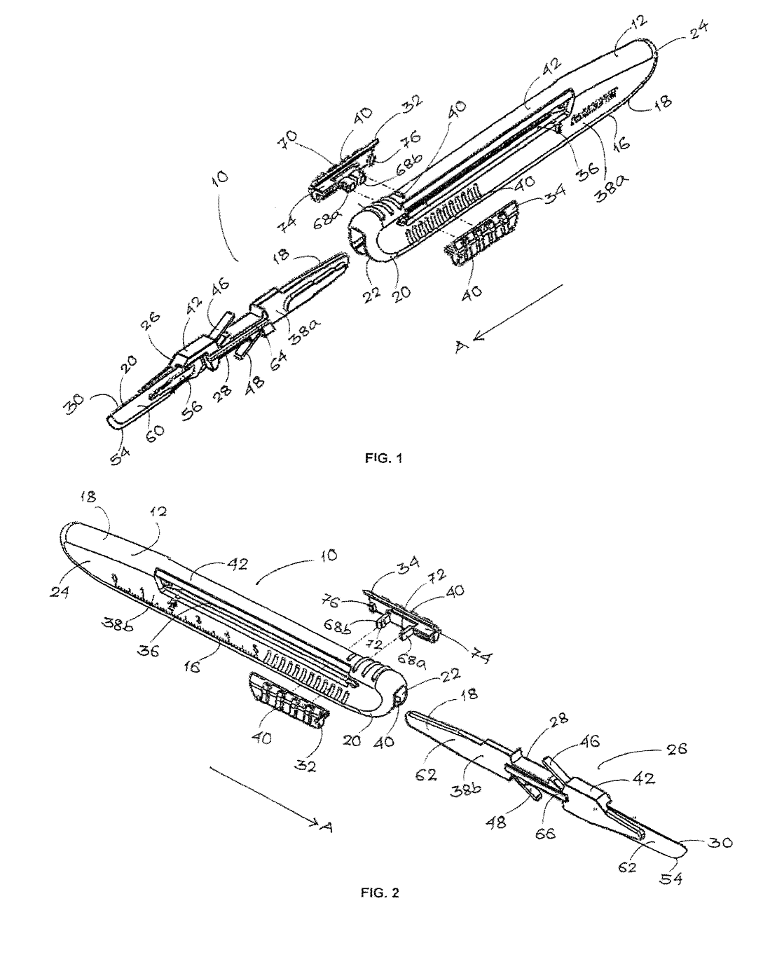

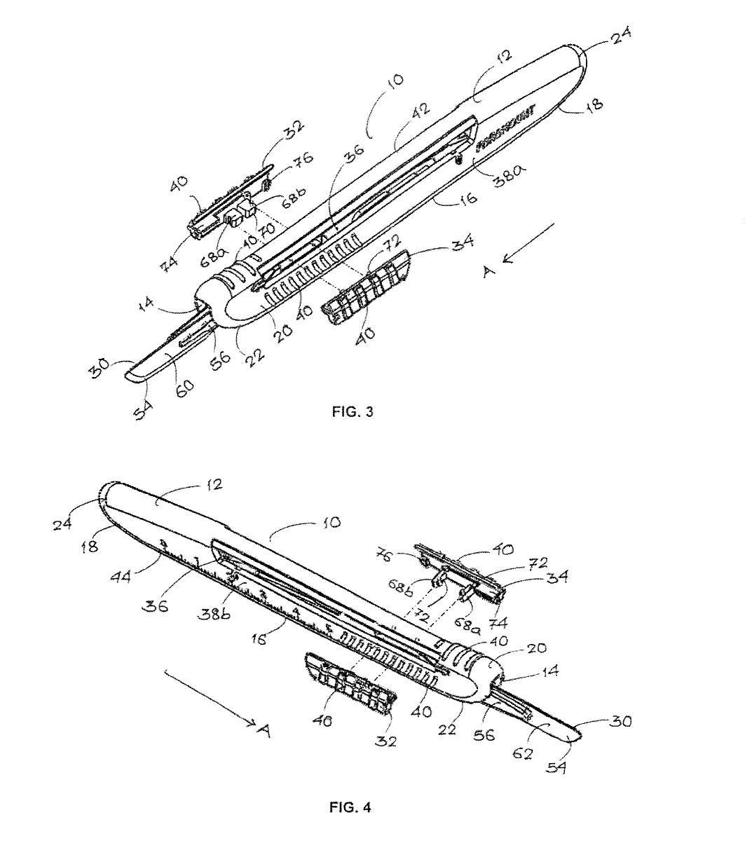

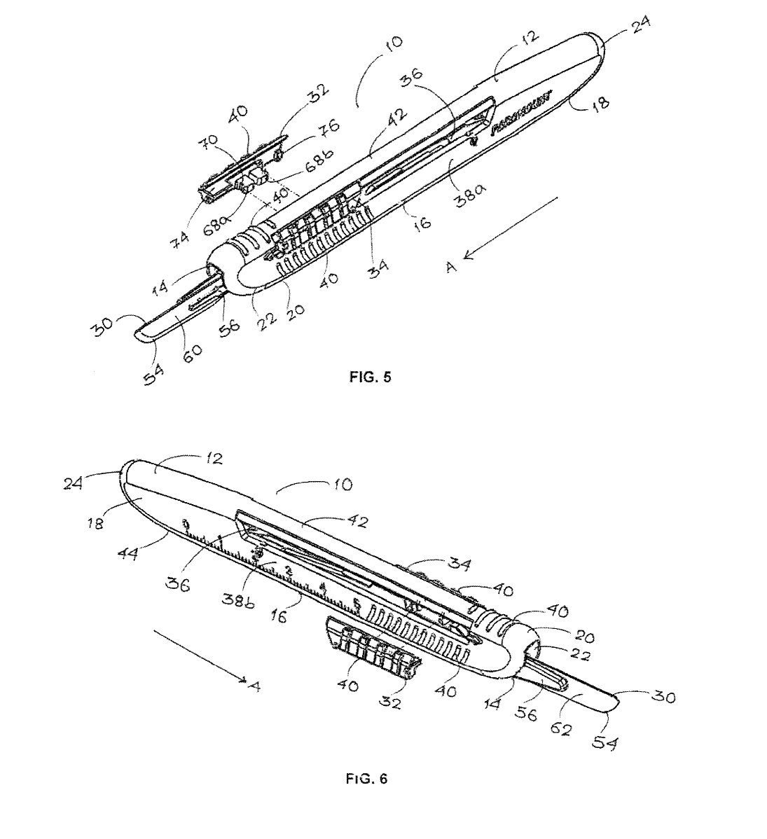

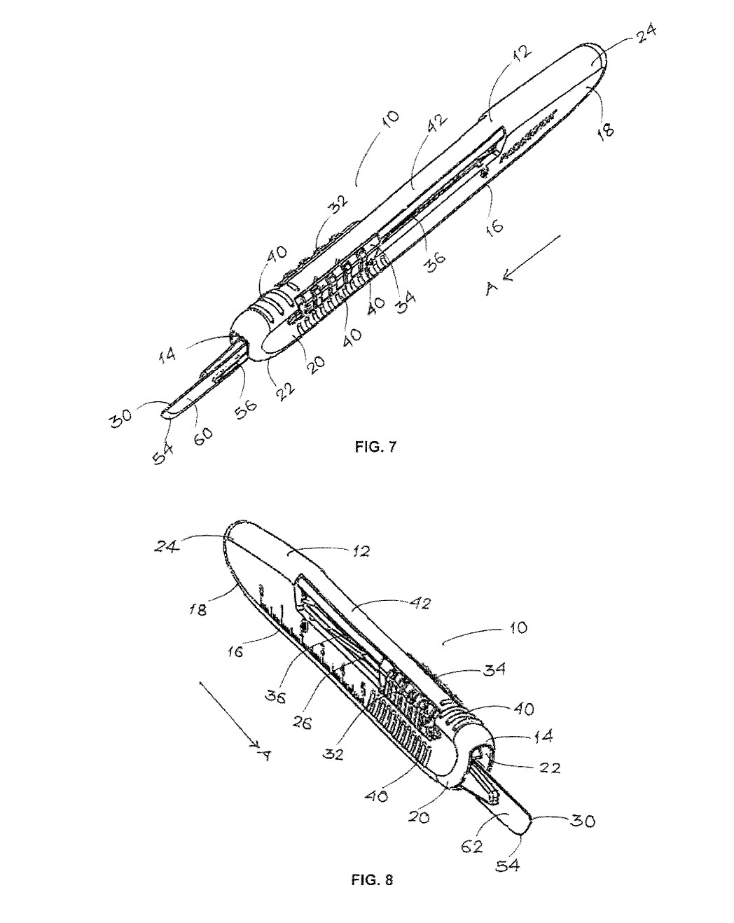

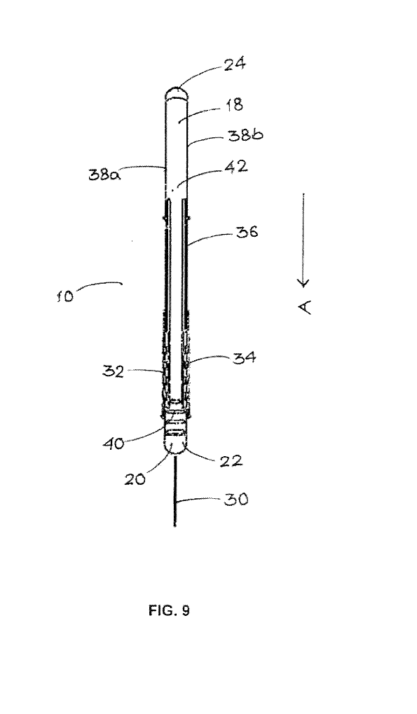

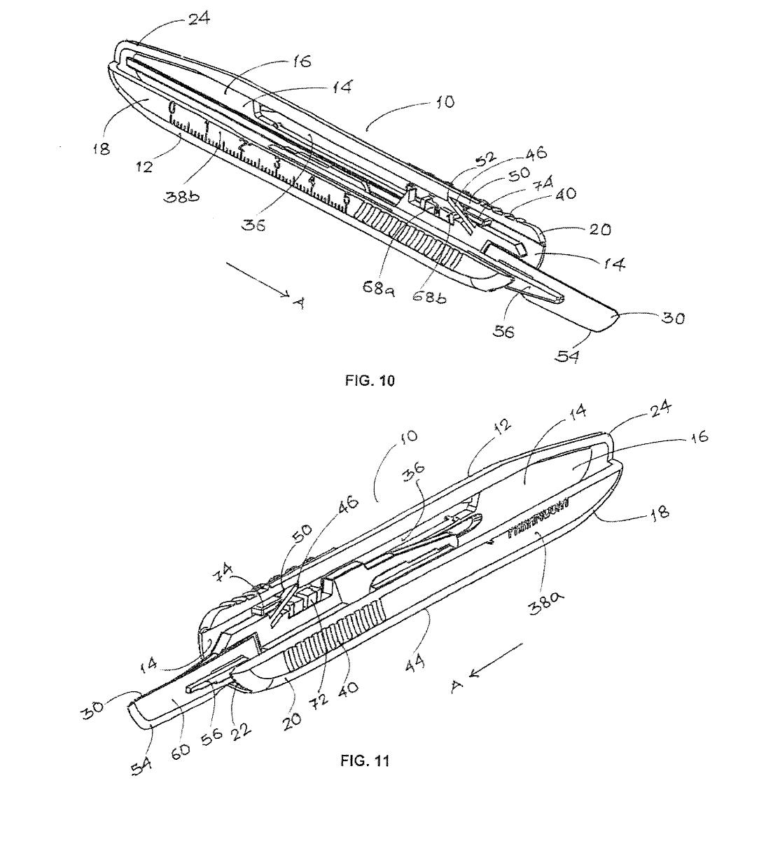

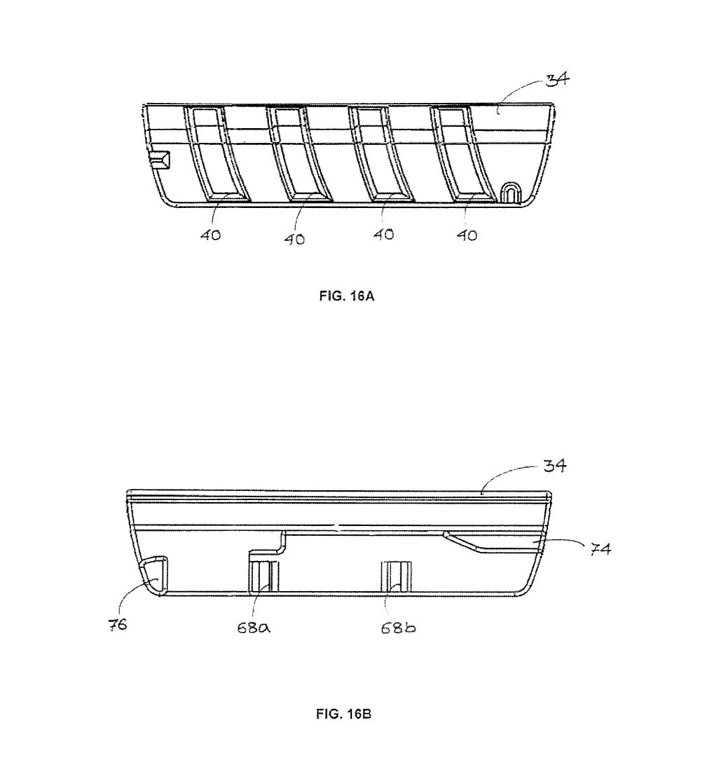

Referring now in detail to the FIGS. 1 to 18 wherein like reference numerals represent like parts throughout the views illustrated, the retractable universal safety scalpel 10 comprises a handle 12 having a proximal 18 section and a distal section with a longitudinally extending bore 14 defining a housing 16, the housing 16 having a opening 22 at the distal 20 end and a closed end 24 at the proximal 18 section of the said handle 12, a blade carrier element 26 having mid-section 28, proximal 18 end and a distal 20 end on which is secured a blade 30 slidably mounted within said housing 16 being capable of getting exposed from the said opening 22 at the distal 20 section of said housing 16; first and second slider elements secured to said blade carrier element 26 through a recess 36 formed in the said handle 12 extending from the forward end closer to the said opening 22 to the rear end closer to the distal 20 section of the said handle 12 in a slidable arrangement with the said housing 16 and extending externally in an axial direction—A of said housing 16 to move said blade carrier 26 moving the said blade 30 from an un-exposed pre-use position to an exposed in-use position and to an after-use position capable of covering and locking permanently the said blade 30 completely within the said housing 16 of the said handle 12.

As can be seen FIGS. 1 to 11 and 18 the handle 12 defining a housing 16 comprises a generally rectangular shape with substantially rounded outer edges. The distal 20 section of the handle 12 is configured as an opening 22 and the proximal 18 section forms the rear end of the handle 12. The handle 12 is provided with a bore 12 extending in an axial direction—A from opening 22 at the distal 20 end towards the rear end at the proximal 18 section. The side walls of the handle 12 is provided with a recess 36 from the forward end closer to the opening 22 to the rear end closer to the distal 20 section of the handle 12. The recess 36 is designed as a cut out. It extends over the substantial part of handle 12 region, and stretches over the greater part of the blade carrier element 26 comprising the blade 30. As can be seen in FIGS. 1 to 11, the recess 36 is designed such that a substantially equal wall strength results in the entire safety scalpel 10, which is especially advantageous in the manufacture of the retractable universal safety scalpel 10 by an injection molding process for example out of plastic or other suitable matter. One skilled in the art will understand that the recesses can also be designed having other shapes and in particular smaller, for instance as circular hollows or other geometric forms.

The handle 12 also comprises finger grip formation on the top 42 and side wall surfaces 38a, 38b of the handle 12 at the distal 20 section thereof. The grip formation includes one or more ribs 40 forming frictional contact with an individual's fingers. The invention embodies alternate positions of one or more similar ribs 40 along the outer surface of the handle 12. The ribs 40 facilitate gripping the scalpel 10 by the surgeon during use.

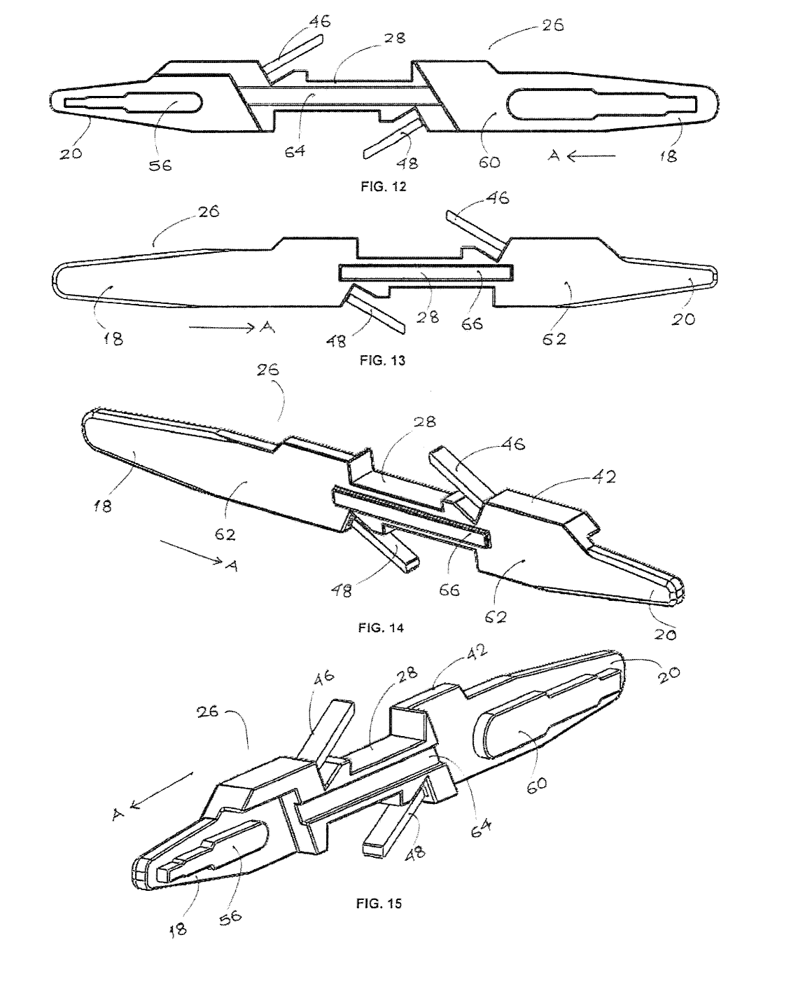

As shown in FIGS. 12 to 15, the blade carrier element 26 comprises several important aspects of the invention in that the distal 20 end forming the forward part and the proximal 18 end forming the rear part is widened compared with mid section 28 so that top 42 and bottom 44 surfaces of the forward and rear part slidably engage the top 42 and bottom 44 surfaces of the bore 12 within the housing 16. The blade carrier element 26 combine with first 32 and second 34 slider elements being connected to the recess 36 provide substantially all the support between the cover housing 16 and the blade 30 secured to the blade carrier element 26. The blade carrier element 26 is also provided with first 46 and second 48 flexible latching elements extending in angular opposing direction adjoining the mid section 28 of the blade carrier element 26. The first 46 and second 48 flexible elements slidably engage with the first 32 and second 34 slider elements when secured in the housing 16. Further, the distal 20 section of the recess 36 has a increased cross-sectional area 50 having dimension of abutment 52 such that the first latching element abuts against the abutment when the blade 30 is in its forward open position being exposed out of the said opening 22.

Further, the blade carrier element 26 includes a blade 30. The blade 30 may be integrally formed with the blade carrier element 26 or may be detachable member capable of being secured with the said blade carrier element 26. The blade 30 includes a cutting edge 54 at the distal 20 end thereof and designed to mate with the mating protrusion 56 positioned near the distal 20 end of the blade carrier element 26. As such, the blade 30 defines an elongated aperture such as a slot 58 near the distal 20 end which in the mating position with the blade carrier element 26 is received onto the mating protrusion 56 positioned near the distal 20 end at the front face of the blade carrier element 26. The mating protrusion 56 formed onto the blade carrier element 26 is thus adapted to securely engage the aperture of the blade 30. During assembly of the blade carrier 26, the blade 30 is fixed to the blade carrier 26 by aligning the aperture of the blade 30 and mating protrusion 56 of the blade carrier element 26. Once the blade 30 aperture is received onto the mating protrusion 56 of the blade carrier 26; it prevents completely the blade 30 from disengaging with the blade carrier element 26. Alternatively, the blade 30 can be connected to the blade carrier element 26 through insert moulding, such that the blade carrier element 26 is formed around the blade 30 during the manufacturing process. Further, the blade 30 has a similar front and back face. The blade 30 can be made of a variety of suitable materials including, but not limited to, carbon and/or stainless material.

The blade carrier element 26 is configured to slide longitudinally within the bore 12 and to receive and support a variety of different profiles of scalpel blades 30. The blade carrier element 26 is elongate in shape and has front and back face having a mid section 28 and proximal 18 and distal 20 ends. The front and back face of the blade carrier element 26 are dissimilar, wherein the mid section 28 of the blade carrier element 26 has a longitudinally extending substantially bar shaped groove 64 on the front face. As opposed to this, the mid section 28 of the blade carrier element 26 on the back face is provided with a longitudinally extending substantially bar shaped projection 66. The inner surface of the handle 12 defining housing 16 is designed to receive the said groove 64 and projection 66 configured on the front and back faces of the blade carrier element 26 in cooperating projection 66 and groove 64 respectively from the inner side wall of the handle 12.

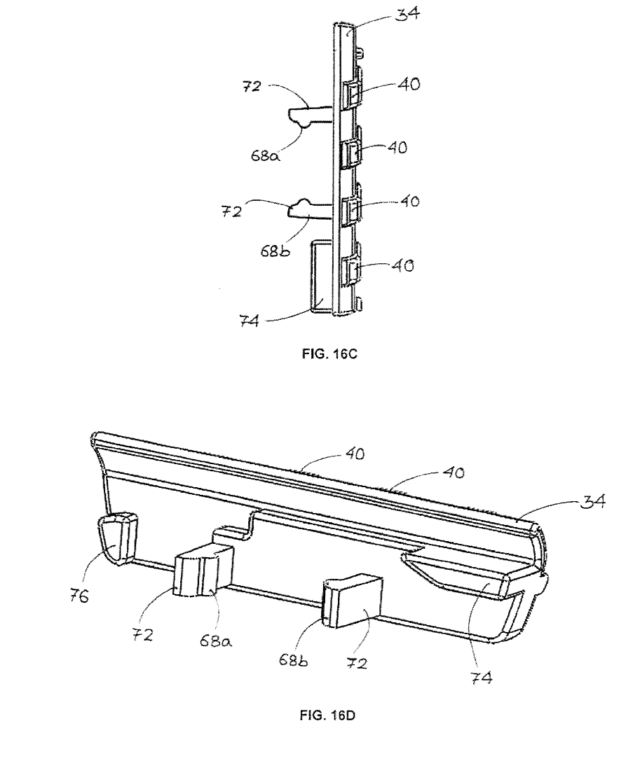

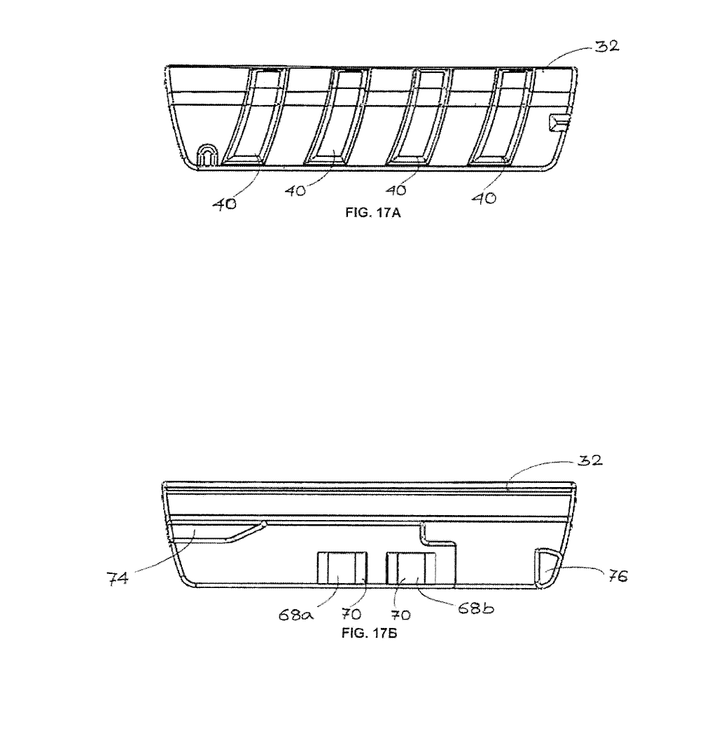

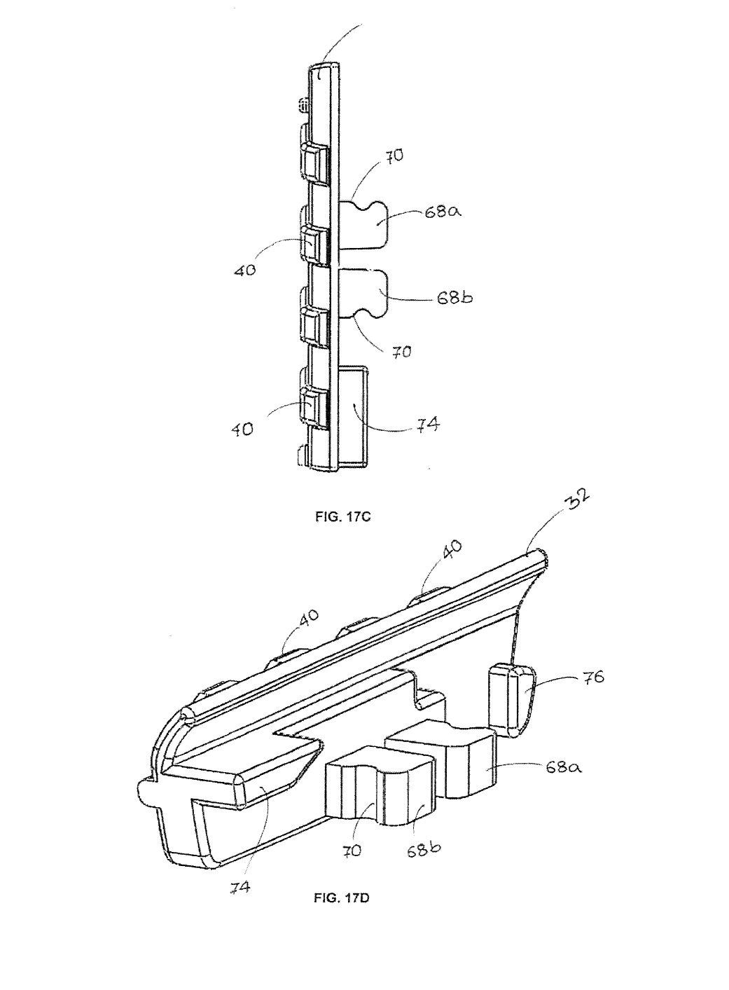

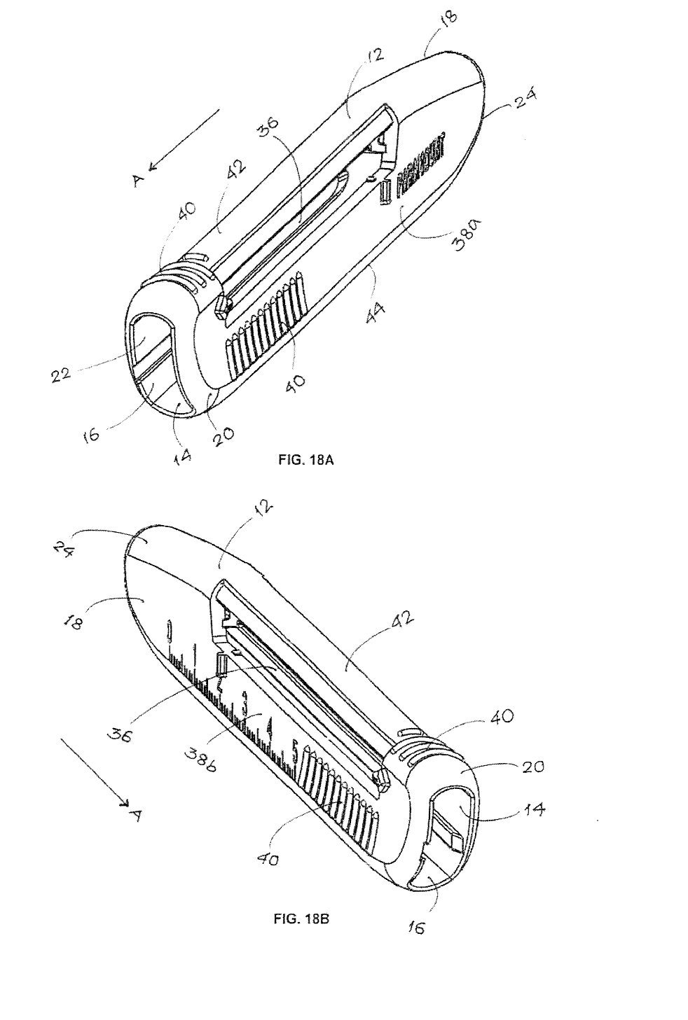

As can be seen in FIGS. 16 and 17 the first slider element 32 on its inner surface in the mid section 28 bottom part is provided with at least two projections 68a, 68b extending outwardly therefrom, the projections 68a, 68b each having lock formation 70 at its free end being capable of locking secure with the second 34 slider element when positioned in the recess 36 provided in the handle 12. Likewise the second slider 34 element on its inner surface in the mid-section 28 bottom part is provided with at least two projections 68a, 68b extending outwardly therefrom, the projections 68a, 68b each having catch formations 72 at its free end are adapted to receive the projections 68a, 68b of the first 32 slider element when positioned in the recess 36 provided in the handle 12. When secured in the recess 36 of the handle 12 the first and second 34 slider elements locks onto each other securing the blade carrier 26 within the bore 12 in the housing 16 and the blade carrier 26 can be slid forwards and rearwards by manually operating the first 32 and second 34 slider element holding the scalpel handle 12 with the use of fingers.

The inner surface of the slider elements 32, 34 is also provided with a forward tab 74 and rear tab 76. The forward tab 74 is positioned near the distal 20 ends thereof and the rear tab 76 is positioned near the proximal 18 end of the slider elements 32, 34. The forward tab 74 is adapted to engage the first 46 and second 48 flexible latching elements when the blade 30 is in-use position. The forward tab 74 prevents the blade carrier element 26 from moving the blade 30 forwardly beyond open position. Similarly, the rear tab 76 is adapted to engage the blade carrier element 26 from moving the blade 30 rearwardly beyond the closed position 24. Alternatively, the rear tab 76 prevents the blade carrier element 26 from moving the blade 30 rearwardly beyond the fully and/or permanently locked position. The first 32 and second 34 slider elements are received in the space defined by a laterally recessed 36 surface on the handle 12 forming a rim structure which extends about and forms the margin of said slot in a movably sliding arrangement. The outer surface of the first 32 and second 34 slider elements is also provided with finger grip formations in the forms of ribs 40, which facilitate a frictional contact with an individual's fingers facilitating grip support. The retractable universal safety scalpel 10 can be easily used/handled by both a right-handed as well as a left-handed person without having to alter the structure of the scalpel 10 and blade 30 due to their symmetrical configuration.

The retractable universal safety scalpel 10 constructed in accordance with present invention embodies the use of various sizes of blades 30 which may be permanently or reasonably attached to the said blade carrier 26, depending on the nature of the use of the scalpel 10.

The handle 12 is preferably formed entirely of a plastic material and, in this form, includes a recess 36 extending in an axial direction—A in between the proximal 18 and distal 20 section of the handle 12.

By holding the handle 12 in the palm of the individual's hand and placing the fingers along the ribs 40 provided on the handle 12 and slider elements i.e., the first 32 and second 34 sliding elements may be advanced from pre-use position in the direction of distal 20 section to in-use position exposing the blade 30 out of the housing 16 of the handle 12 for use. The degree of control and movement of the slider elements 32, 34 corresponds with the degree of the blade 30 being exposed out of distal 20 section of the handle 12. In transitioning between the two positions, it will be appreciated that the handle 12 can be operated with only one hand, thus freeing the other hand for other work.

After use and when it is desirable to dispose of the scalpel 10, the slider elements 32, 34 may be advanced in the direction of proximal 18 section of the handle 12 into its permanently locked position. To accomplish this, the handle 12 is once again placed in the user's hand allowing the fingers to advance the sliding elements in the direction of proximal 18 section of the handle 12 to a permanently locked position. In the permanently locked position engaging means as provided in the blade 30 carrier element 26 and slider elements 32, 34 prompt to blocking rearward return movement of the sliding elements. Consequently, the sliding elements are permanently locked, covering the blade 30, whereby individuals are protected from inadvertent, casual and non-intentional contact with the blade 30.

Once the scalpel 10 is in permanently locked position, it is impossible to use the scalpel 10 again. The scalpel blade 30 is thus held in an extremely safe locked inoperative position for disposal, thereby avoiding the possibility of any so-called sharps injuries. The scalpel 10 may then be handled for further disposal without the danger attendant to an exposed blade 30.

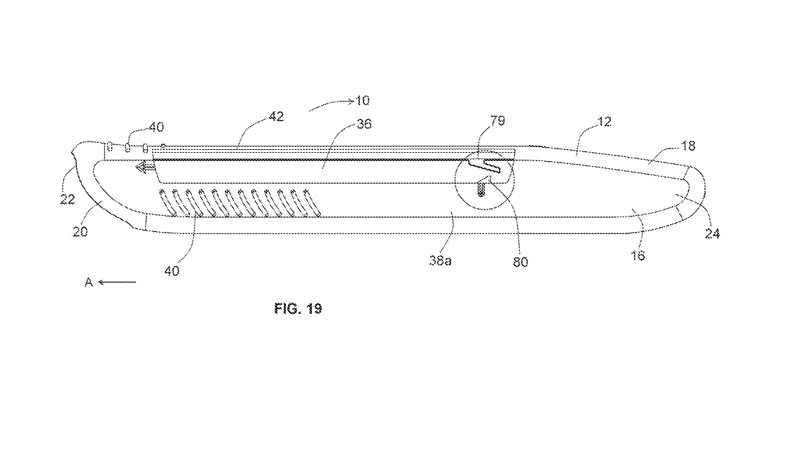

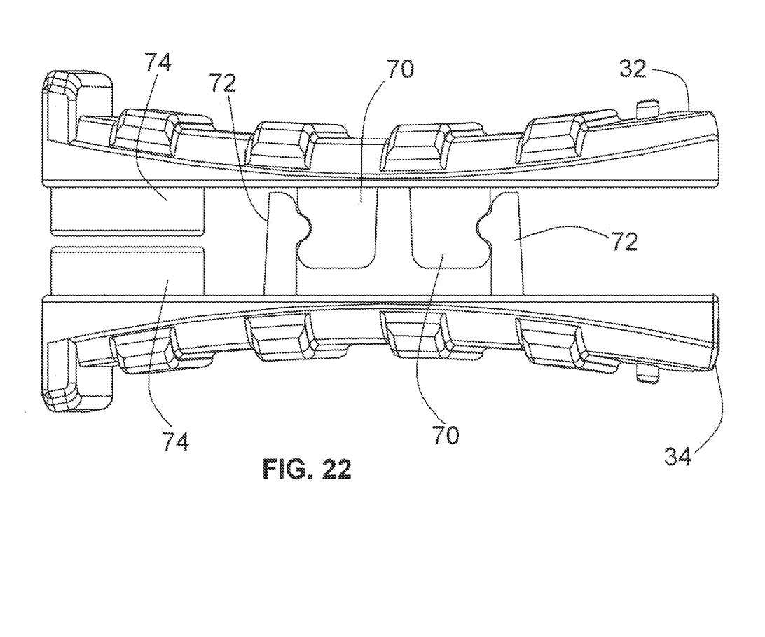

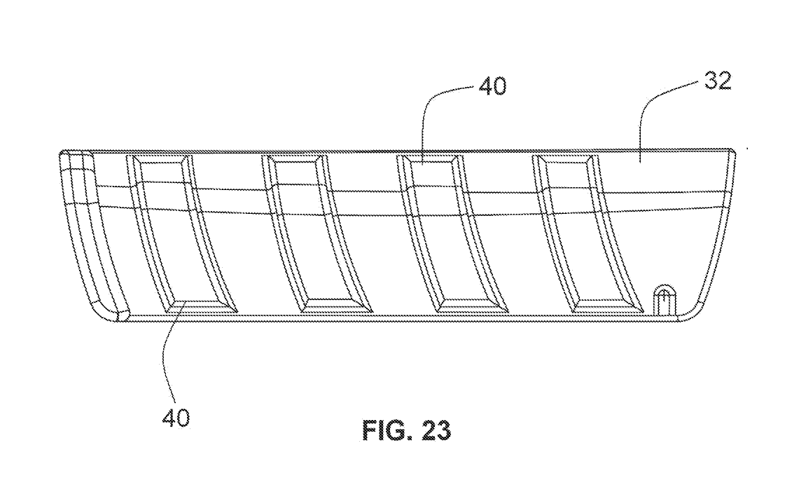

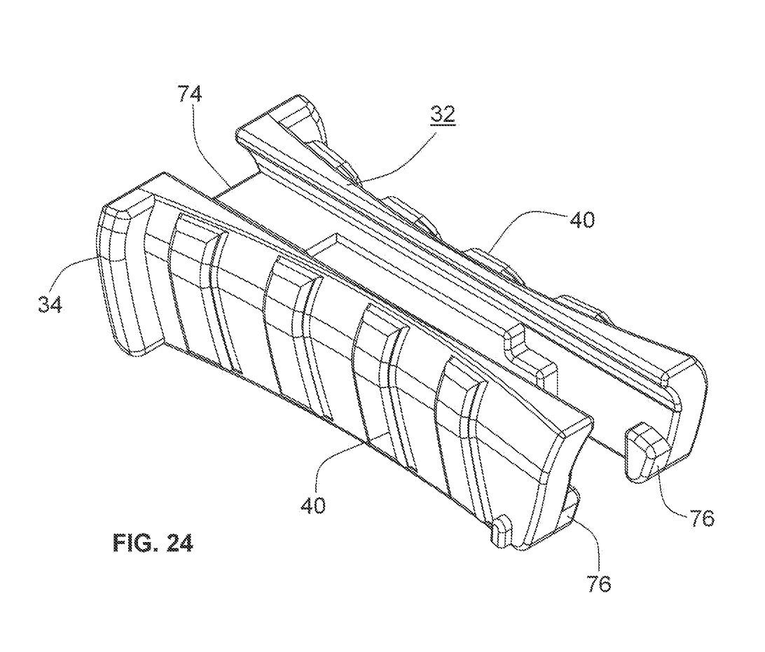

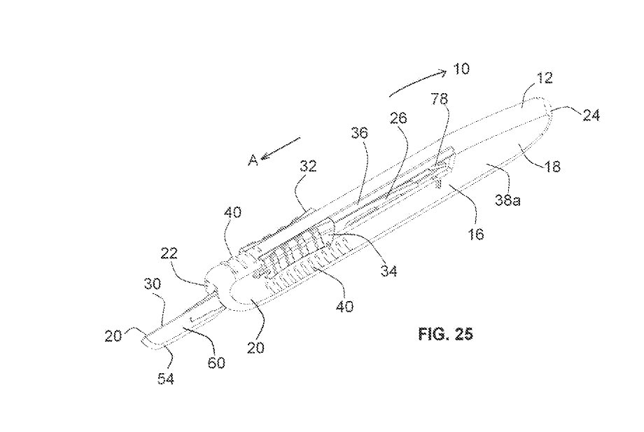

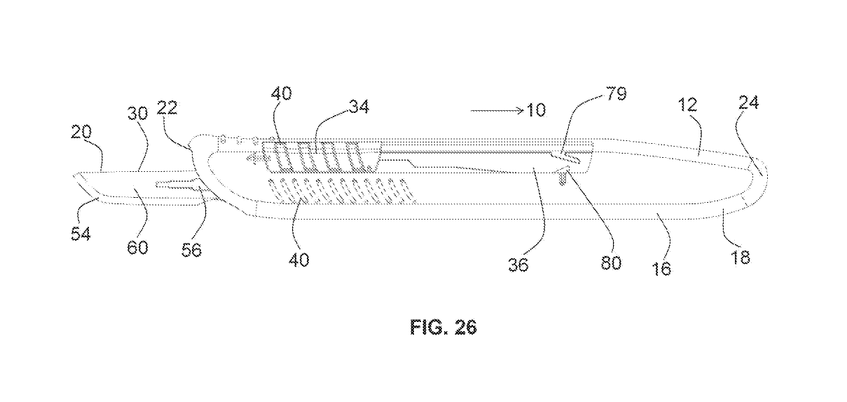

In one embodiment, as shown in FIGS. 19 to 26, the housing 16 is provided with a bore 14 which holds the blade carrier element 26. The blade carrier element 26 is slidably arranged in the bore 14 from an un-exposed pre-use condition to an exposed in-use position and to an after-use locked disposal position where the blade is permanently locked. The safety scalpel 10 is provided with plurality of locking means to ensure locking of the blade carrier element in various positions in particular to ensure locking of the blade carrier element 26 in the housing in use position and to ensure permanent locking of the blade carrier element 26 with the blade 30 in a retracted position when the safety scalpel 10 is ready for disposal. As a result of the permanent disposal locking position, the safety of handlers during disposal is ensured. Similarly, the easy locking transfer between the in-use position to permanent locking position ensures that transfer of the safety scalpel 10 during operation does not result in any accidental nick or cut to the user or handler.

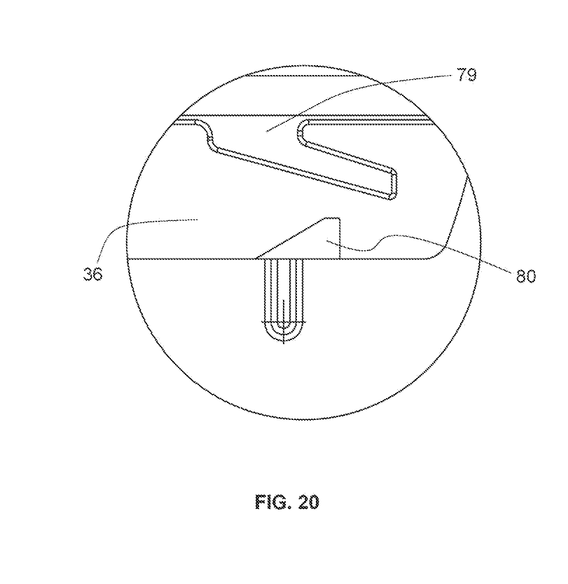

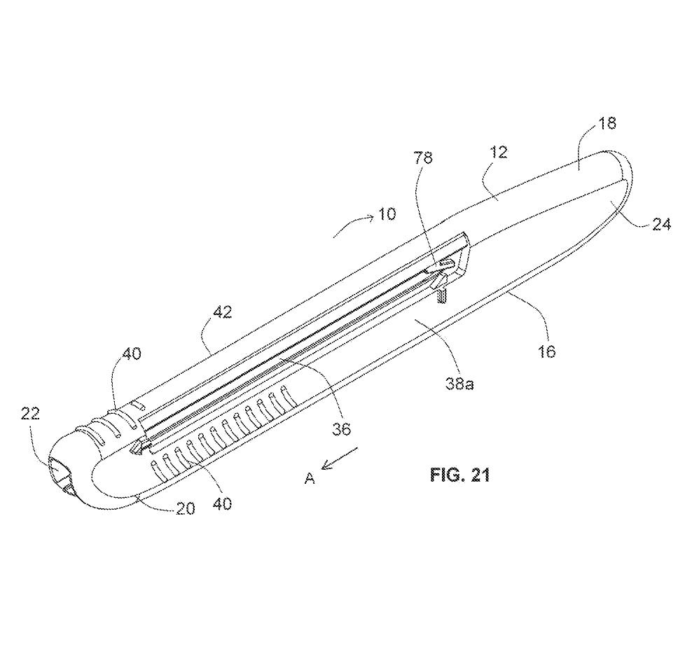

The locking means comprise at least a pair of locking members 78 formed integrally in the housing 16 provided in the housing, in particular in the region of the recess 36 proximal to the closed end 24 of the housing 16, being adapted to engageably receive the slider elements 32, 34 and retain them therein securely in a locked position. The said pair of locking members 78 shown in greater detail in FIG. 20 include at least one locking finger 79 and at least one locking tab 80 adapted to engageably receive the slider elements 32, 34 in particular the projections 68a, 68b and tabs 74, 76 provided thereon to ensure a permanent locking position. After final completion of the surgical procedure, the blade carrier element 26 is pushed back to a final permanent locking position with the help of slider elements 32, 34 where locking means including locking members 78 engage with the projections 68a, 68b and tabs 74, 76 provided on the slider elements 32, 34 to ensure permanent locking. In this final stop position, the blade carrier element 26 with the blade 30 cannot be released accidentally, thereby ensuring complete safety of the user.

The name “Paramount” appearing in the drawings is the trade mark of Paramount Surgimed Ltd.

Thus, the present invention provides for a retractable universal safety scalpel 10 having safety features which once activated cannot be deactivated and remains protective through disposal; perform reliably and easy to use and is thus safe and effective during surgical procedure and afterwards.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, from the foregoing description, it will be apparent to one of ordinary skill in the art that many changes and modifications can be made thereto without departing from the spirit or scope of the invention as set forth herein.

Accordingly, it is not intended that the scope of the foregoing description be limited to the exact description set forth above, but rather that such description be construed as encompassing all of the features of patentable novelty that reside in the present invention, including all the features and embodiments that would be treated as equivalents thereof by those skilled in the relevant art. Thus, it is intended that the scope of the present invention herein disclosed should not be limited by the particular disclosed embodiments described above but should be determined only by a fair reading of the claims appended below.

1. A retractable universal safety scalpel comprising a handle having a proximal section and a distal section with a longitudinally extending bore defining a housing;
a blade carrier element having mid-section, proximal end and a distal end on which is secured a blade slidably mounted within said housing being capable of getting exposed from the end of distal section of said housing;
first and second slider elements secured to said blade carrier element through a recess formed in said handle extending from the forward end closer to said opening to the rear end closer to the distal section of said handle in a slidable arrangement with said housing and extending externally in an axial direction of said housing to move said blade carrier moving said blade from an un-exposed pre-use position to an exposed in-use position and to an after-use position capable of covering and locking permanently said blade member completely within said housing of said handle;
a plurality of locking means is provided in the said housing to enable locking of the blade in an un-exposed pre-use position to an exposed in-use position and to an after-use position capable of covering and locking permanently said blade member wherein said housing being provided with lock cooperating mechanisms at three positions thereon corresponding to pre-use, in-use and post-use disposal stages, said lock cooperating mechanisms cooperating with the respective locking means provided thereon.

2. The retractable universal safety scalpel as claimed in claim 1, wherein the housing having an opening at the distal end and a closed end at the proximal section of said handle.

3. The retractable universal safety scalpel as claimed in claim 1, wherein said handle at its sidewalls is provided with a recess from the forward end closer to the opening to the rear end closer to the distal section of said handle.

4. The retractable universal safety scalpel as claimed in claim 1, wherein said handle is provided with finger grip formation on the top and side wall surfaces of the handle at the distal section thereof, said grip formation includes one or more ribs forming frictional contact with an individual's fingers.

5. The retractable universal safety scalpel as claimed in claim 1, wherein said blade defines an elongated aperture near the distal end thereof.

6. The retractable universal safety scalpel as claimed in claim 1, wherein said blade carrier element comprises a mating protrusion positioned near the distal end thereof such that the said mating protrusion is adapted to engage the aperture of the blade.

7. The retractable universal safety scalpel as claimed in claim 1, wherein said blade carrier element comprises a front and back face having a mid-section and proximal end forming the forward part and distal end forming the rear part, such that the front and back faces are dissimilar in profile.

8. The retractable universal safety scalpel as claimed in claim 7, wherein said distal end forming the forward part and proximal end forming the rear part is widened compared with said mid-section of the blade carrier element, such that top and bottom surfaces of the forward part slidably engage the top and bottom surfaces of the bore within said housing.

9. The retractable universal safety scalpel as claimed in claim 7, wherein said mid-section has a longitudinally extending substantially bar shaped groove on the front face, such that when the blade carrier element is slidably arranged in said housing said groove is received in a mating projection formed on the inner side wall of said handle and wherein said mid-section has a longitudinally extending substantially bar shaped projection on the back face, such that when the blade carrier element is slidably arranged in said housing said groove is received in a mating groove formed on the inner side wall of said handle.

10. The retractable universal safety scalpel as claimed in claim 1, wherein said blade carrier element is provided with first and second flexible latching elements extending generally in angular opposing directions ad-joining said mid-section of the blade carrier element, such that when secured in the housing said latching elements slidably engage with the said first and second slider elements.

11. The retractable universal safety scalpel as claimed in claim 1, wherein said recess has an increased cross-sectional area having dimension of abutment such that the first latching element abuts against said abutment when the blade is in its forward open position being exposed out of said opening.

12. The retractable universal safety scalpel as claimed in claim 1, wherein said first slider element on its inner surface in the mid-section bottom part is provided with at least two projections extending outwardly therefrom, said projections each having lock formation at its free end.

13. The retractable universal safety scalpel as claimed in claim 1, wherein said second slider element on its inner surface in the middle section bottom part is provided with at least two projections extending outwardly therefrom, the projections each having catch formations at its free end, such that said catch formations are adapted to receive the projections of the first slider element when positioned in the recess provided in said handle.

14. The retractable universal safety scalpel as claimed in claim 12, wherein said slider elements comprises on its inner surface a forward tab positioned near the distal end thereof and a rear tab positioned near the proximal end thereof.

15. The retractable universal safety scalpel as claimed in claim 1, wherein the locking means comprise at least a pair of locking members provided in the housing, in particular in the region of the recess proximal to the closed end of the housing, being adapted to engagably receive the slider elements and retain them therein securely in a locked position.
